(19) United States
(12) Patent Application Publication
(10) Pub. No.: US 2011/0153020 A1
(43) Pub. Date: Jun. 23, 2011

(54) Anterior Lumbar Interbody

(75) Inventors: Mahmoud F. Abdelgany, Rockaway, NJ (US); Kevin Sichler, West Orange, NJ (US)

(73) Assignee: CUSTOM SPINE, INC., Parsippany, NJ (US)

(21) Appl. No.: 12/641,229

(22) Filed: Dec. 17, 2009

(51) Int. Cl. A61F 2/44

(52) U.S. Cl. 623/17.16

(57) ABSTRACT

A system having an expandable intervertebral fusion implant assembly for insertion between two vertebrae bodies. The system includes an anterior lumbar interbody component having an upper component and a lower component, each having an inner and outer surface, pivotally connected through a hinge. A distracting component includes a wedge having a superior and inferior surface that inserts between the upper and lower components of the anterior lumbar interbody component. The outer surface of the upper and the lower component contact endplates of two adjacent vertebral bodies.

### BACKGROUND

1. Technical Field

The embodiments herein generally relate to medical devices, and, more particularly, to medical devices for the spine.

2. Description of the Related Art

One of the most costly health problems to society involves back pain and pathology of the spine, which can be caused by several factors. These problems can affect individuals of all ages and can result in great suffering to victims.

Upon identification of these abnormalities, surgery may be required to correct the problem. One method of correction is spinal fusion, wherein vertebrae are fixed together to prevent movement and maintain a space originally occupied by an intervertebral disk. During a spinal fusion, a prosthetic implant or spinal implant is inserted into the intervertebral space. Although this procedure may result in some minor loss and flexibility in the spine, due to the relatively large number of vertebrae, the minor loss of mobility is typically acceptable.

Most commonly, a manufactured implant biologically compatible with the body and the vertebrae, is made of a synthetic material. Several compositions and geometries of such implants have been is utilized, ranging from simple blocks of material to carefully shaped implants, with varying success. There, has been an extensive number of attempts in developing an acceptable prosthetic implant that can be used to replace an intervertebral disk and yet maintain the stability of the intervertebral disk spaced between adjacent vertebrae. Examples of some conventional systems are described in U.S. Pat. No. 6,849,093 and U.S. Pat. No. 7,326,248, the complete disclosures of which, in their entireties, are herein incorporated by reference.

While many types of synthetic prosthetic devices have been proposed, the success ratio has been low and the surgical procedures have been complicated and often traumatic to the patient. Although intervertebral fusion is widely used, drawbacks to their use include a reduced physiologic range of motion and other fusion related complications such as degeneration of adjacent discs and destabilization of the functional spinal unit. As a result, alternative treatments with fewer complications, but similar efficacy to fusion, are desirable.

### SUMMARY

In view of the foregoing, an embodiment herein provides a system having an expandable intervertebral fusion implant assembly for insertion between two vertebrae bodies. The system includes an anterior lumbar interbody component having an upper component and a lower component, the upper component and the lower component each having an inner and outer surface. The upper and lower components are pivotally connected through a hinge. A distracting component including a wedge having a superior and inferior surface inserts between the upper and lower components of the anterior lumbar interbody component. The outer surface of the upper and the lower component contact endplates of two adjacent vertebral bodies.

The outer surface of the upper component and the lower component each include a vertebrae position device at a first end proximal to the hinge. The upper and the lower component include textured area terminating at the vertebrae position device and a rib proximal to the first end. The distracting component includes a securing component continuous with the wedge, wherein the securing component extends substantially perpendicular from the wedge. The securing component includes a first and second fixation hole traversing the securing component, wherein the first and second fixation holes are positioned on an upper section and a lower section of the securing component, wherein the wedge is positioned between the upper section and the lower section. The securing component includes a bendable metal to accommodate curvature of vertebrae bodies. The distracting component has at least one alignment fin proximal to the wedge. The upper and the lower component each have at least one fin guide positioned complementary to the at least one alignment fin proximal to the wedge. The superior and inferior surfaces of the wedge are angled. The second end of the upper and the lower component is contoured to the shape of the securing component. The anterior lumbar interbody component is substantially D-shaped.

Another embodiment provides an expandable intervertebral fusion implant apparatus for insertion between two vertebrae bodies including an anterior lumbar interbody component having an upper component and a lower component. The upper component and the lower component each have an inner and outer surface, wherein the upper and lower components are pivotally connected through a hinge. A distracting component has a wedge having a superior and inferior surface, wherein the wedge inserts between the upper and lower components of the anterior lumbar interbody component. A second end of the upper and the lower component each have a bone screw guide, wherein the bone screw guide is angled.

The distracting component includes a securing component continuous with the wedge via an extension bar, wherein the securing component extends substantially perpendicular from the wedge. The securing component is substantially square shaped having a top edge and a bottom edge, wherein the top edge and the bottom edge are substantially aligned with the outer surface of the upper component and the lower component. The securing component includes a first and second fixation hole traversing the securing component, wherein the first and second fixation holes are positioned diagonally on the securing component. The wedge is positioned substantially in the middle of the securing component. The fixation holes are angled complementary to the angled bone screw guides. A second end of the upper and the lower component is contoured to the shape of the securing component. The superior and inferior surfaces of the wedge are angled.

Yet another embodiment provides an expandable intervertebral fusion implant assembly for insertion between two vertebrae bodies including an anterior lumbar interbody component having an upper component and a lower component. The upper and lower components are pivotally connected through a hinge. A distracting component includes a wedge that inserts between the upper and lower components of the anterior lumbar interbody component, a vertebrae position device at a first end proximal to the hinge, a rib proximal to the first end, a securing component continuous with the wedge, a first and second fixation hole traversing the securing component, at least one alignment fin proximal to the wedge, and at least one fin guide positioned complementary to the at least one alignment fin proximal to the wedge.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1A is a schematic diagram illustrating a perspective view of an anterior lumbar interbody component according to a first embodiment herein;

FIG. 1B is a schematic diagram illustrating a top view of the anterior lumbar interbody component of FIG. 1A according to a first embodiment herein;

FIG. 1C is a schematic diagram illustrating a front view of the anterior lumbar interbody component of FIG. 1A in the closed position according to a first embodiment herein;

FIG. 1D is a schematic diagram illustrating a front view of the anterior lumbar interbody component of FIG. 1A in the open position according to a first embodiment herein;

FIG. 2A is a schematic diagram illustrating a perspective view of a distracting component according to a first embodiment herein;

FIG. 2B is a schematic diagram illustrating a side view of the distracting component of FIG. 2A according to a first embodiment herein;

FIG. 2C is a schematic diagram illustrating a front view of the distracting component of FIG. 2A according to a first embodiment herein;

FIG. 3A is a schematic diagram illustrating a perspective view of an assembly comprising the anterior lumbar interbody component of FIGS. 1A through 4D partially engaging the distracting component of FIGS. 2A through 2C according to a first embodiment herein;

FIG. 3B is a schematic diagram illustrating a perspective view of the assembly comprising the anterior lumbar interbody component of FIGS. 1A through 4D fully engaging the distracting component of FIGS. 2A through 2C according to a first embodiment herein;

FIG. 3C is a schematic diagram illustrating a side view of an assembly comprising an anterior lumbar interbody component fully engaging a 20-degree inclined distracting component according to a first embodiment herein;

FIG. 3D is a schematic diagram illustrating a side view of an assembly comprising an anterior lumbar interbody component fully engaging a 10-degree inclined distracting component according to a first embodiment herein;

FIG. 3E is a schematic diagram illustrating a side view of an assembly comprising an anterior lumbar interbody component fully engaging a 10-degree inclined bent distracting component according to a first embodiment herein;

FIG. 4A is a schematic diagram illustrating a side view of a spinal column comprising the assembly of FIG. 3B according to an embodiment herein;

FIG. 4B is a schematic diagram illustrating a top view of a spinal column comprising the assembly of FIG. 3B according to an embodiment herein;

FIG. 5A is a schematic diagram illustrating a perspective view of an assembly comprising an anterior lumbar interbody component fully engaging a distracting component according to a second embodiment herein;

FIG. 5B is a schematic diagram illustrating a side view of the assembly of FIG. 5A according to a second embodiment herein;

FIG. 5C is a schematic diagram illustrating a front view of the assembly of FIG. 5A according to a second embodiment herein;

FIG. 6A is a schematic diagram illustrating a perspective view of an assembly comprising an anterior lumbar interbody component fully engaging a distracting component according to a third embodiment herein;

FIG. 6B is a schematic diagram illustrating a front view of the assembly of FIG. 6A according to a third embodiment herein;

FIG. 6C is a schematic diagram illustrating a side view of the assembly of FIG. 6A according to a third embodiment herein;

FIG. 6D is a schematic diagram illustrating a perspective view of the anterior lumbar interbody component of the assembly of FIG. 6A according to a third embodiment herein;

FIG. 6E is a schematic diagram illustrating a side view of the anterior lumbar interbody component of FIG. 6D according to a third embodiment herein;

FIG. 7A is a schematic diagram illustrating a perspective view of an assembly comprising an anterior lumbar interbody component fully engaging a distracting component according to a fourth embodiment herein;

FIG. 7B is a schematic diagram illustrating a front view of the assembly of FIG. 7A according to a fourth embodiment herein;

FIG. 7C is a schematic diagram illustrating a side view of the assembly of FIG. 7A according to a fourth embodiment herein;

FIG. 7D is a schematic diagram illustrating a perspective view of the anterior lumbar interbody component of the assembly of FIG. 7A according to a fourth embodiment herein;

FIG. 7E is a schematic diagram illustrating a side view of the anterior lumbar interbody component of FIG. 7D according to a fourth embodiment herein;

FIG. 8A is a flow diagram illustrating a first method according to an embodiment herein;

FIG. 8B is a flow diagram illustrating a second method according to an embodiment herein;

FIG. 8C is a flow diagram illustrating a third method according to an embodiment herein; and

FIG. 8D is a flow diagram illustrating a fourth method according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

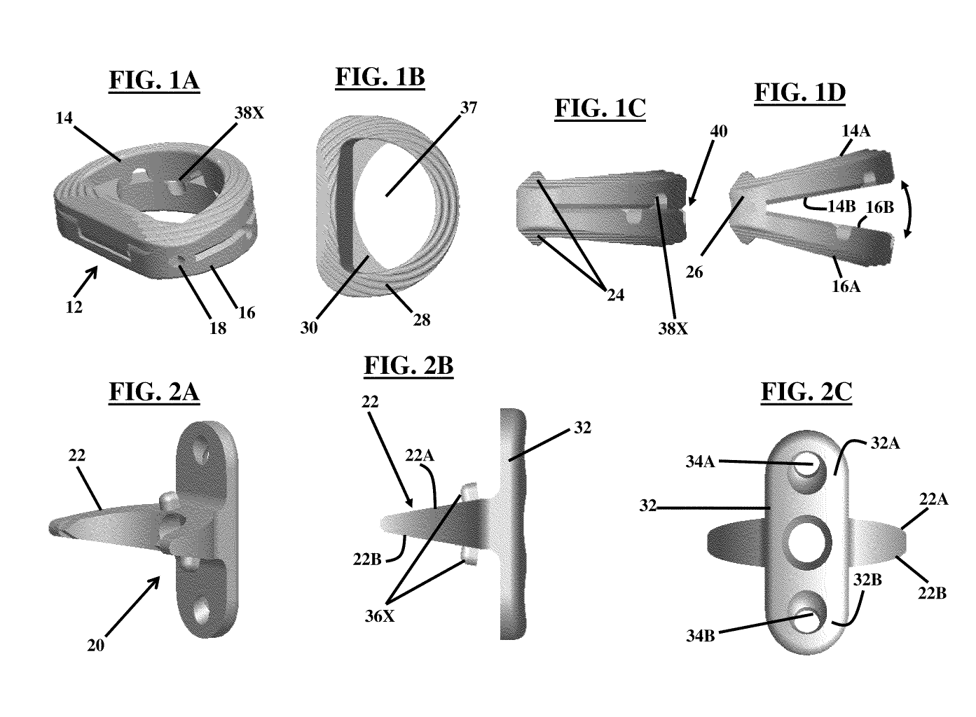

The embodiments herein provide an expandable intervertebral fusion implant for insertion between two vertebrae; comprising of an open superior (upper) and inferior (lower) component, that are pivotally connected through a hinge, and distracting component that comprises of a wedge that is adapted to engage/move apart the upper and lower components to contact the endplates of the vertebral bodies. The device allows the surgeon to adapt/adjust the angle of the implant to help accommodate the intervertebral disc space with a more concise fit. Referring now to the drawings, and more particularly to FIGS. 1A through 8D, where similar reference characters denote corresponding features consistently throughout the figures, there are shown preferred embodiments.

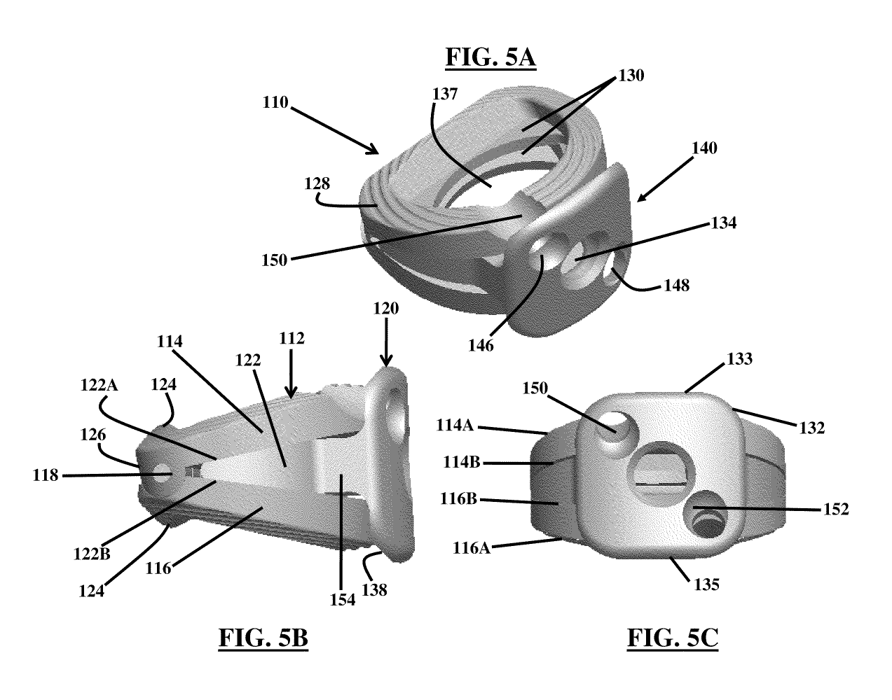

A first embodiment herein, illustrated in FIGS. 1A through 4B, provides an expandable intervertebral fusion implant system 10 for insertion between two vertebrae bodies 1000, as best illustrated in FIGS. 4A and 4B. Referring to FIGS. 1A through 3E, the system 10 includes an anterior lumbar interbody component 12 having an upper component 14 and a lower component 16 each having an inner and outer surface 14A, 14B, 16A, 16B. The upper and lower components 14, 16 are pivotally connected through a hinge 18 allowing opening and closing for positioning as described herein. A distracting component 20 includes a wedge 22 having a superior 22A and inferior surface 22B. The wedge 22 is configured to insert between the upper 14 and lower 16 components of the anterior lumbar interbody component 12 based on the “open and close” movement allowed by the hinge 18. This positioning and movement allows the outer surfaces 14A, 16A of the upper 14 and lower 16 components to contact endplates 1002 of two adjacent vertebral bodies 1000, as best illustrated in FIGS. 4A and 4B.

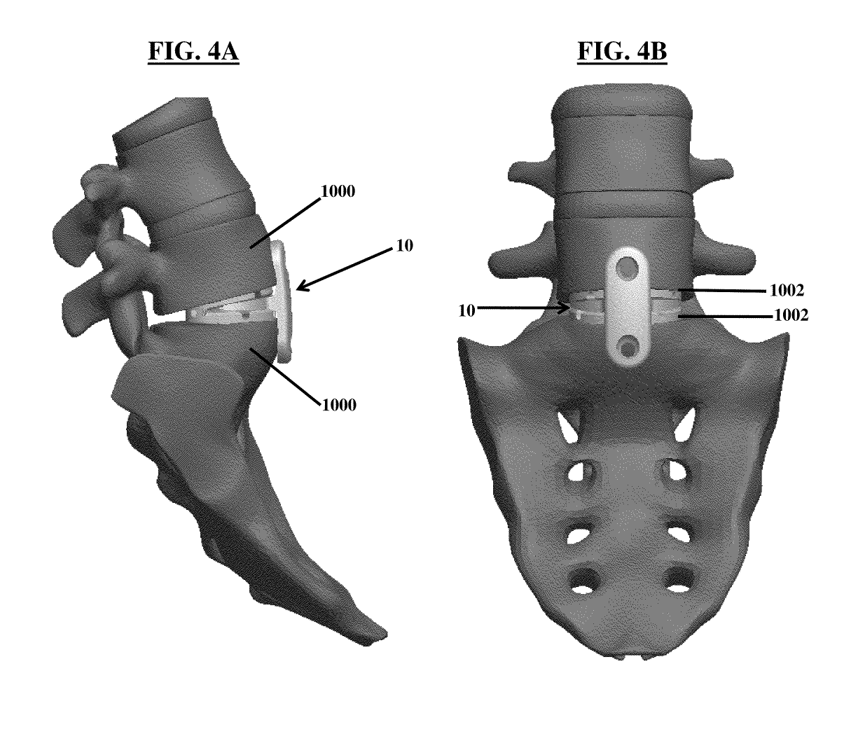

The outer surfaces 14A, 16A of the upper component 14 and the lower component 16 each include a vertebrae position device 24 at a first end 26 proximal to the hinge 18. The vertebrae position device 24 allows for an improved fit and reduction of movement of the assembly 10. The addition of a textured feature 28 on the outer surfaces 14A, 16A of the upper component 14 and the lower component 16, further reduces movement of the system 10 upon installation. Further, referring to FIGS. 1B and 3A, the upper 14 and lower 16 component each comprise of a rib 30 proximal to the first end 26 and a graft containment area 37. Rib 30 provides the anterior lumbar interbody component 12 torsional stiffness.

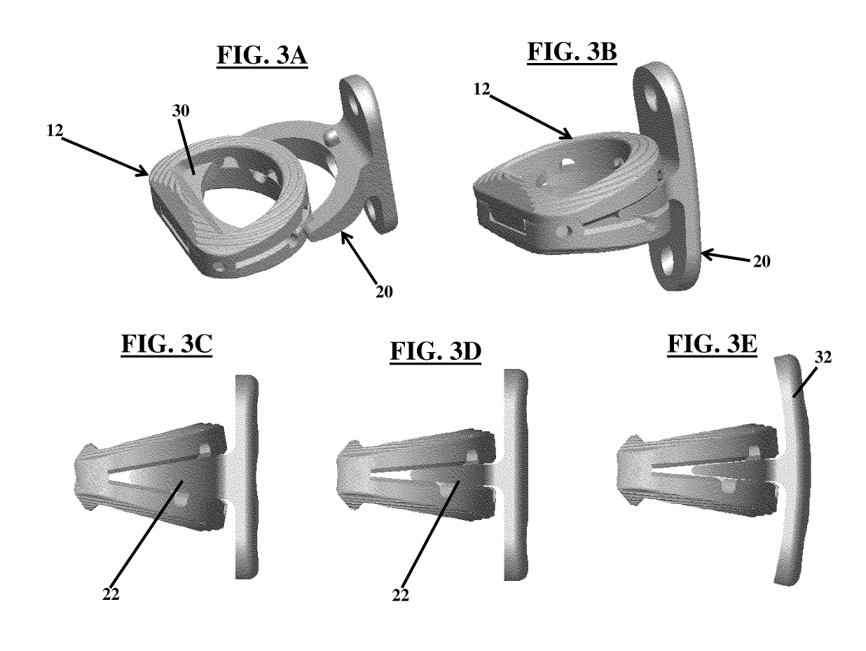

As further shown in FIGS. 2A through 2C, the distracting component 20 includes a securing component 32 continuous with the wedge 22, and extending substantially perpendicular to the wedge 22. The securing component 32 has a first and second fixation hole 34A, 34B traversing the securing component 32, and positioned on an upper section 32A and a lower section 32B of the securing component 32. The fixation holes 34A, 34B are position so as to align with adjacent vertebrae 1000, as best illustrated in FIGS. 4A, 4B, and to accommodate fixation screws (not shown) for securing the distracting component 20 to the vertebrae 1000. The wedge 22 is positioned between the upper section 14 and the lower section 16 of the securing component 32, thereby allowing attachment with even pressure on the vertebrae. As the wedge 22 is introduced in between the upper section 14 and the lower section 16, the components are distracted, forcing a line-to-line fit with the endplates 1002 of the vertebral disc space and the outer surfaces 14a and 16a. The anterior lumbar interbody component 12 accommodates a variety of angles to allow for optimal placement and fit. The securing component 32 is preferably made of a metallic material, which may be bent to accommodate the curvature of vertebrae bodies 1000 and thereby, improve movement, as illustrated in FIG. 3E.

The distracting component 20 includes at least one alignment fin 36X proximal to the wedge 22 positioned complementary to at least one alignment fin guide 38X on the upper 14 and/or lower 16 component of the interbody component 12. It is appreciated that the exact number and location of alignment fins 36X and complementary alignment fin guides 38X could vary without diverting from the basic concept of a complementary locating and “fit” means for combining the anterior lumbar interbody component 12 and distracting component 20.

Preferably, the superior 22A and inferior 22B surfaces of the wedge 22 are configured to form an angle of approximately 10.0 degrees to 20.0 degrees. Referring to FIGS. 3C and 3D, a 10° and a 20° wedge 22 are separately illustrated. One skilled in the art would recognize that the wedge “angle” could be configured in other various degrees. Most commonly, the wedge 22 would be of one size for each distracting component 20 and would be designated as required by the patient's procedure as recognized by a surgeon. It will be recognized by those of skill in the art that the hinge 18 would “open” allowing movement of the upper 14 and lower 16 component so as to accommodate the “shape” of the wedge 22 upon insertion. Further, a second end 40 of the upper 14 and lower 16 component is contoured to the shape of the securing component 32, which comprises bendable metal. The anterior lumbar interbody component 12 is most commonly a “D” shape so as to complement the meeting of the second end 40 of the upper 14 and lower 16 component with the securing component 32 of the distracting component 20.

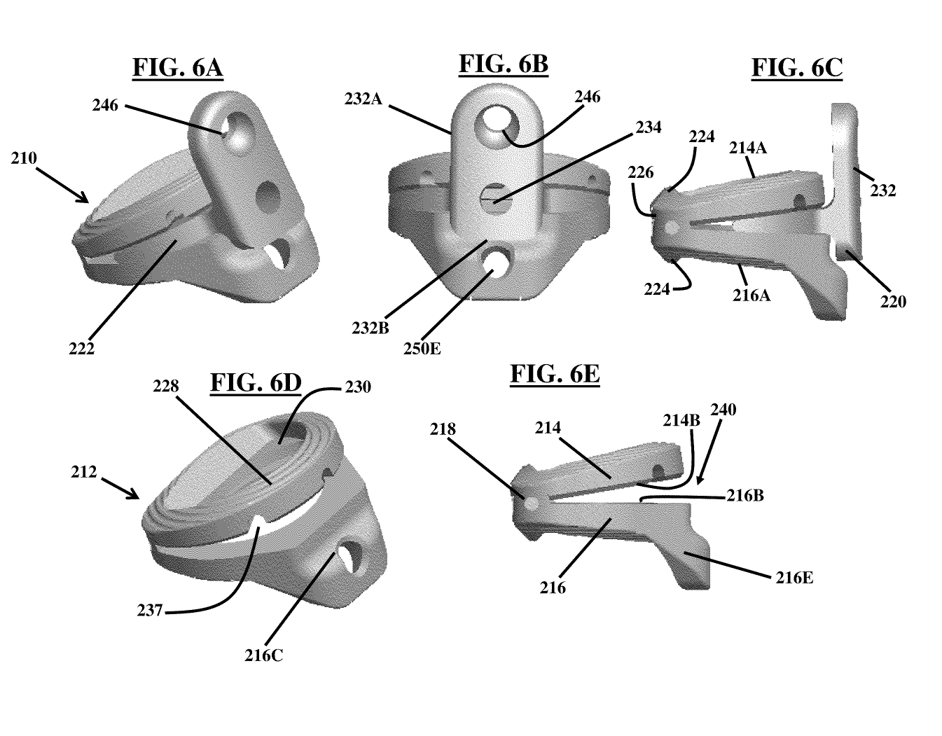

Referring to FIGS. 5A through 5C, a second embodiment herein is illustrated comprising an expandable intervertebral fusion implant apparatus 110 for insertion between two vertebrae bodies (e.g., vertebrae 1000 of FIGS. 4A and 4B). The apparatus 110 includes an anterior lumbar interbody component 112 comprising an upper component 114 and a lower component 116, each having an inner surface 114B, 116B and outer surface 114A, 116A; the upper 114 and lower 116 components are pivotally connected through a hinge 118. A distracting component 120 includes a wedge 122 having a superior 122A and inferior 122B surface. The wedge 122 is configured to insert between the upper 114 and lower 116 components of the anterior lumbar interbody component 112. A second end 140 of each of the upper 114 and the lower 116 component includes an angled bone screw guide 150, 152.

The distracting component 120 further includes a securing component 132 continuous with the wedge 122 via an extension bar 154, wherein the securing component 132 extends substantially perpendicular to the wedge 122. The securing component 132 is substantially square shaped having a top edge 133 and a bottom edge 135, which are substantially aligned with the outer surface 114A, 116A of upper component 114 and the lower component 116. The securing component 132 includes a fixation hole 134 traversing the securing component 132. The fixation hole 134 is positioned centrally on the securing component 132. Additionally, a pair of corner fixation holes 146, 148 are positioned on opposite corners of the securing component 132 such that the holes 134, 146, 148 are diagonally positioned on the securing component 132. The holes 146, 148 are aligned with the respective angled bone screw guides 150, 152. The wedge 122 is positioned between substantially centrally from the back end 138 of the securing component 132. As with the previous embodiment, the second end 140 of the upper 114 and the lower 116 component is contoured to the shape of the securing component 132.

Similar to the previous embodiment, the superior 122A and inferior 122B surface of the wedge 122 form an angle of approximately 10° to 20°. Further, the wedge 122 would be of one size for each distracting component 120 and would be designated as required by the patient's procedure as recognized by a surgeon. It will be recognized by those of skill in the art that the hinge 118 would “opens” allowing movement of the upper 114 and lower 116 component so as to accommodate the “shape” of the wedge 122 upon insertion.

The outer surfaces 114A, 116A of the upper component 114 and the lower component 116 each include a vertebrae position device 124 at a first end 126 proximal to the hinge 118. The vertebrae position device 124 allows for an improved fit and reduction of movement of the assembly 110. The addition of a textured feature 128 on the outer surfaces 114A, 116A of the upper component 114 and the lower component 116, further reduces movement of the assembly 110 upon installation. The upper 114 and lower 116 component each comprise a graft containment guide 130 proximal to the first end 126 and a graft containment area 137.

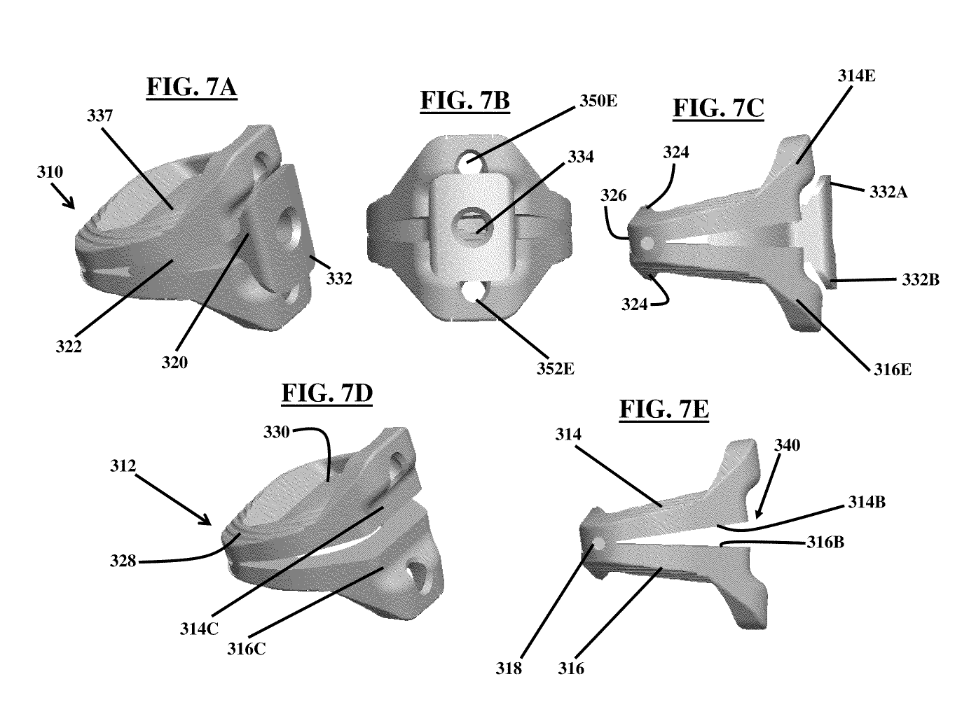

In FIGS. 6A through 6E, with reference to FIGS. 1A through 5C, a third embodiment herein is illustrated comprising an expandable intervertebral fusion implant apparatus 210 for insertion between two vertebrae bodies (e.g., vertebrae 1000 of FIGS. 4A and 4B). The apparatus 210 includes an anterior lumbar interbody component 212 comprising an upper component 214 and a lower component 216 each having an inner and outer surface 214A, 214B, 216A, 216B, and pivotally connected through a hinge 218 located at a first end 226 of the interbody component 212. A second end 240 of the interbody component 212 includes an extended portion 216E connected to the lower component 216, wherein the extended portion 216E comprises an angled bone screw guide 250E.

The outer surfaces 214A, 216A of the upper component 214 and the lower component 216 each include a vertebrae position device 224 at a first end 226 proximal to the hinge 218. The vertebrae position device 224 allows for an improved fit and reduction of movement of the assembly 210. The addition of a textured feature 228 on the outer surfaces 214A, 216A of the upper component 214 and the lower component 216, further reduces movement of the system 210 upon installation. The upper 214 and lower 216 component each comprise of a rib 230 proximal to the first end 226 and a graft containment area 237. Rib 230 provides the anterior lumbar interbody component 212 torsional stiffness.

A distracting component 220 includes a securing component 232 continuous with a wedge 222, wherein the securing component 232 extends substantially perpendicular to the wedge 222. The securing component 232 includes a centrally positioned fixation hole 234 traversing the securing component 232. Upon attachment of the apparatus 210, a screw (not shown) would contact the compacted bone graft material that is inserted into the graft containment area 237. Since the bone graft material allows the bone to regenerate, the screw would then become anchored in the newly formed regenerated bone. Additionally, an angled fixation hole 246 is located on the upper portion 232A of the securing component 232 to secure the securing component 232 to a vertebrae 1000 (as illustrated in the previous embodiment in FIGS. 4A and 4B).

The extended lower portion 216E extending from the lower component 216, has increased thickness of the bone screw guide 250E to allow additional depth to secure a screw (not shown) traversed therein. Further, the position of the extended lower portion 216E defines a contour 216C to the shape of the lower portion 232B of the securing component 232 allowing additional support. Moreover, a screw (not shown) fits in the fixation hole 246 when the apparatus 210 is attached to the vertebrae 1000.

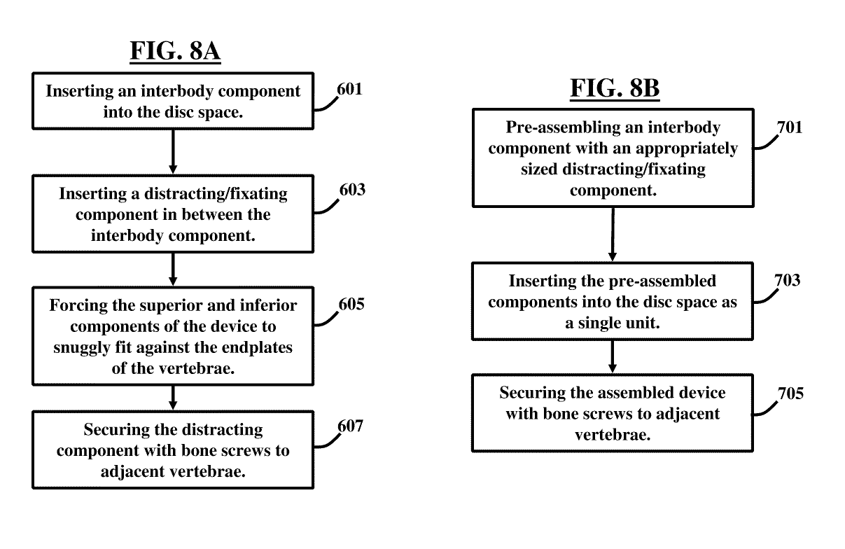

In FIGS. 7A through 7E, with reference to FIGS. 1A through 6E, a fourth embodiment herein is illustrated having an expandable intervertebral fusion implant apparatus 310 for insertion between two vertebrae bodies (e.g., vertebrae 1000 of FIGS. 4A and 4B). The apparatus 310 includes an anterior lumbar interbody component 312 comprising an upper component 314 and a lower component 316 each having an inner and outer surface 214A, 214B, 216A, 216B, and pivotally connected through a hinge 318 located at a first end 326 of the interbody component 312. A second end 340 of the interbody component 312 includes extended portions 314E, 316E extending from the upper component 314 and lower component 316, respectively, and having angled bone screw guides 350E, 352E.

The outer surfaces 314A, 316A of the upper component 314 and the lower component 316 each include a vertebrae position device 324 at a first end 326 proximal to the hinge 318. The vertebrae position device 324 allows for an improved fit and reduction of movement of the assembly 310. The addition of a textured feature 328 on the outer surfaces 314A, 316A of the upper component 314 and the lower component 316, further reduces movement of the system 310 upon installation. The upper 314 and lower 316 component each comprise of a rib 330 proximal to the first end 326 and a graft containment area 337. Rib 330 provides the anterior lumbar interbody component 312 torsional stiffness.

A distracting component 320 includes a securing component 332 continuous with a wedge 322, wherein the securing component 332 extends substantially perpendicular to the wedge 322. The securing component 332 includes a centrally positioned fixation hole 334 traversing the securing component 332. Upon attachment of the apparatus 310, a screw (not shown) would contact the compacted bone graft material that is inserted into the graft containment area 337. Since the bone graft material allows the bone to regenerate, the screw would then become anchored in the newly formed regenerated bone.

Each extended lower portion 314E, 316E extends from the upper component 314 and lower component 316, respectively, and allows increased thickness of the bone screw guides 350E, 352E to allow additional depth to secure a screw (not shown) inserted therein. Further, the position of the extended portions 314E, 316E define a contour 314C, 316C to the shape of the upper portion 332A and lower portion 332B of the securing component 332, allowing additional support.

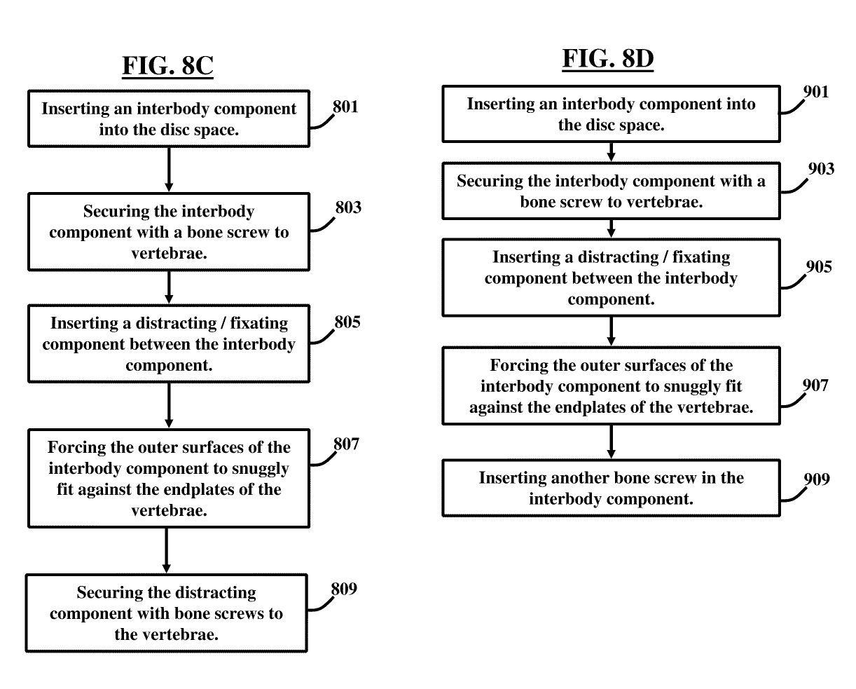

The assembly 10, 110 is intended to be used in the anterior lumbar spine as an intervertebral fusion device. FIGS. 8A and 8B, with reference to FIGS. 1A through 5C, illustrate different methods for implantation of the assembly 10, 110. Referring to FIG. 8A, the interbody component 12 is inserted (601) into the disc space and thereafter, the distracting/fixating component 20 is inserted (603) in between the interbody component 12. This method proceeds by forcing (605) the superior and inferior components of the device to snuggly fit against the endplates 1002 of the vertebrae 1000. Finally, the method involves securing (607) the distracting component 20 with bone screws (not shown) to adjacent vertebrae 1000.

Referring to FIG. 8B, the interbody component 12 is pre-assembled (701) with an appropriately sized distracting/fixating component 20 and inserted (703) into the disc space as a single unit. The assembled device 10 is secured (705) with bone screws (not shown) to adjacent vertebrae 1000.

FIGS. 8C and 8D, with reference to FIGS. 6A through 7E, illustrate alternate methods according to the embodiments herein. Referring to FIG. 8C, an interbody component 212 is inserted (801) into the disc space and is secured (803) with a bone screw through angled bone screw guide 250E to vertebrae 1000. A distracting/fixating component 232 is then inserted (805) between the upper and lower components 214, 216 of the interbody component 212. Thereafter, the surfaces 214A, 216A of the interbody component 212 is forced to snuggly fit (807) against the endplates 1002 of the vertebrae 1000. Finally, the distracting component 232 is secured (809) with bone screws via hole 246 to the vertebrae 1000.

Referring to FIG. 8D, with reference to FIGS. 7A through 7E, an interbody component 312 is inserted (901) into the disc space and is secured (903) with a bone screw through bone screw guide 350E to vertebrae 1000. The wedge 322 of the distracting component 320 is inserted (905) between the upper and lower components 314, 316 of the interbody component 312. Thereafter, the surfaces 314A, 316A of the interbody component 312 is forced to snuggly fit (907) against the endplates 1002 of the vertebrae 1000. Thereafter, another bone screw is inserted (909) through bone screw guide 352E to secure the interbody component 310 to the vertebrae 1000. The distracting/fixating component 320 that is inserted between the interbody component 312 covers the bone screws that are inserted in bone screw guides 350E, 352E in order to prevent screw back out.

Generally, the embodiments herein provide an expandable intervertebral fusion implant assembly 10, 110, 210, 310 for insertion between two vertebrae bodies 1000 and include an anterior lumbar interbody component 12, 112, 212, 312 comprising an upper component 14, 114, 214, 314 and a lower component 16, 116, 216, 316, wherein the upper 14, 114, 214, 314 and lower 16, 116, 216, 316 components are pivotally connected through a hinge 18, 118, 218, 318. The assembly 10, 110, 210, 310 further includes a distracting component 20, 120, 220, 320 that comprises a wedge 22, 122, 222, 322 that inserts between the upper 14, 114, 214, 314 and lower 16, 116, 216, 316 components of the anterior lumbar interbody component 12, 112, 212, 312. A vertebrae position device 24, 124, 224, 324 is positioned at a first end 26, 126, 226, 326 proximal to the hinge 18, 118, 218, 318. A rib 30, 130, 230, 330 is positioned proximal to the first end 26, 126, 226, 326. A securing component 32, 132, 232, 332 is continuous with the wedge 22, 122, 222, 322, and a first 34A, 146, 246, 334 and second 34B, 148, 234 fixation hole traverses the securing component 32, 132, 232, 332. At least one alignment fin 36X is positioned proximal to the wedge 22, and at least one fin guide 38X is positioned complementary to the at least one alignment fin 36X proximal to the wedge 22.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A system comprising an expandable intervertebral fusion implant assembly for insertion between two vertebrae bodies, said system comprising:
an anterior lumbar interbody component comprising an upper component and a lower component, said upper component and said lower component each having an inner and outer surface, wherein the upper and lower components are pivotally connected through a hinge; and
a distracting component comprising a wedge having a superior and inferior surface, wherein said wedge inserts between said upper and lower components of said anterior lumbar interbody component,
wherein said outer surface of said upper and said lower component contact endplates of two adjacent vertebral bodies.

2. The system of claim 1, wherein said outer surface of said upper component and said lower component each comprise a vertebrae position device at a first end proximal to said hinge.

3. The system of claim 2, wherein said outer surface of said upper component and said lower component comprise a textured area, said texture area terminating at said vertebrae position device.

4. The system of claim 3, wherein said upper and said lower component each comprise a rib proximal to said first end.

5. The system of claim 4, wherein said distracting component comprises a securing component continuous with said wedge, wherein said securing component extends substantially perpendicular from said wedge.

6. The system of claim 5, wherein said securing component comprises a first and second fixation hole traversing said securing component, wherein the first and second fixation holes are positioned on an upper section and a lower section of said securing component, wherein said wedge is positioned between said upper section and said lower section.

7. The system of claim 6, wherein said securing component comprises bendable metal to accommodate curvature of vertebrae bodies.

8. The system of claim 7, wherein said distracting component comprises at least one alignment fin proximal to said wedge.

9. The system of claim 8, wherein said upper and said lower component each comprise at least one fin guide positioned complementary to said at least one alignment fin proximal to said wedge.

10. The system of claim 9, wherein said superior and inferior surface of said wedge are angled.

11. The system of claim 10, wherein a second end of said upper and said lower component is contoured to the shape of said securing component.

12. The system of claim 1, wherein said anterior lumbar interbody component is substantially D-shaped.

13. An expandable intervertebral fusion implant apparatus for insertion between two vertebrae bodies comprising:
an anterior lumbar interbody component comprising an upper component and a lower component, said upper component and said lower component each having an inner and outer surface, wherein the upper and lower components are pivotally connected through a hinge; and
a distracting component that comprises a wedge having a superior and inferior surface, wherein said wedge inserts between said upper and lower components of said anterior lumbar interbody component,
wherein said second end of said upper and said lower component each comprise a bone screw guide, wherein said bone screw guide is angled.

14. The apparatus of claim 13, wherein said distracting component comprises a securing component continuous with said wedge via an extension bar, wherein said securing component extends substantially perpendicular from said wedge.

15. The apparatus of claim 14, wherein said securing component is substantially square shaped having a top edge and a bottom edge, wherein said top edge and said bottom edge are substantially aligned with said outer surface of said upper component and said lower component.

16. The apparatus of claim 15, wherein said securing component comprises a first and second fixation hole traversing said securing component, wherein said first and second fixation holes are positioned diagonally on said securing component, wherein said wedge is positioned substantially in the middle of said securing component.

17. The apparatus of claim 16, wherein said fixation holes are angled complementary to the angled bone screw guides.

18. The apparatus of claim 17, wherein a second end of said upper and said lower component is contoured to the shape of said securing component.

19. The apparatus of claim 18, wherein said superior and inferior surface of said wedge are angled.

20. An expandable intervertebral fusion implant assembly for insertion between two vertebrae bodies comprising:
an anterior lumbar interbody component comprising an upper component and a lower component, wherein said upper and lower components are pivotally connected through a hinge;
a distracting component that comprises a wedge that inserts between said upper and lower components of said anterior lumbar interbody component;
a vertebrae position device at a first end proximal to said hinge;
a rib proximal to said first end;
a securing component continuous with said wedge;
a first and second fixation hole traversing said securing component;
at least one alignment fin proximal to said wedge; and
at least one fin guide positioned complementary to said at least one alignment fin proximal to said wedge.
